(19) United States
(12) Patent Application Publication
(10) Pub. No.: US 2003/0181804 A1
(43) Pub. Date: Sep. 25, 2003

(54) Distributed diagnostic imaging systems

(75) Inventors: Daniel Gagnon, Twinsburg, OH (US); James M. McNally, Montville, OH (US); Michael C. Steckner, Richmond Hts., OH (US); Jacob A. Stolk, Salt Lake City, UT (US); Terence P. Young, Essex (GB)

Correspondence Address: Thomas E. Kocovsky, Jr., FAY, SHARPE, FAGAN, MINNICH & McKEE, LLP, Seventh Floor, 1100 Superior Avenue, Cleveland, OH, 44114-2518, US

(73) Assignee: KONINKLIJKE PHILIPS ELECTRONICS N.V.

(21) Appl. No.: 10/102,327

(22) Filed: Mar. 20, 2002

(51) Int. Cl. A61B 5/05

(52) U.S. Cl. 600/410

(57) ABSTRACT

A plurality of diagnostic scanners (S₁, S₂, . . . , Sₙ) share access to a remote, communal processing center (CP) that performs reconstruction and post reconstruction processing for various modalities. Each of the diagnostic scanners submits a data set to the remote center electronically over the lines (T). An scheduling computer (22) assigns a priority to each of the received data sets and controls a plurality of parallel processors (26₁, 26₂, . . . , 26ₙ) accordingly. The reconstructed image representations are sent electronically back to the address that sent them, or another designated location, for display on a monitor (28₁, 28₂, . . . , 28ₙ, 28_{cf}, 28ᵣ). Upgrades loaded into the remote center are immediately available for all users. Software modifications, hardware adjustments, training services, operations monitoring, and scanner operating services of individual scanners are provided from the remote center.

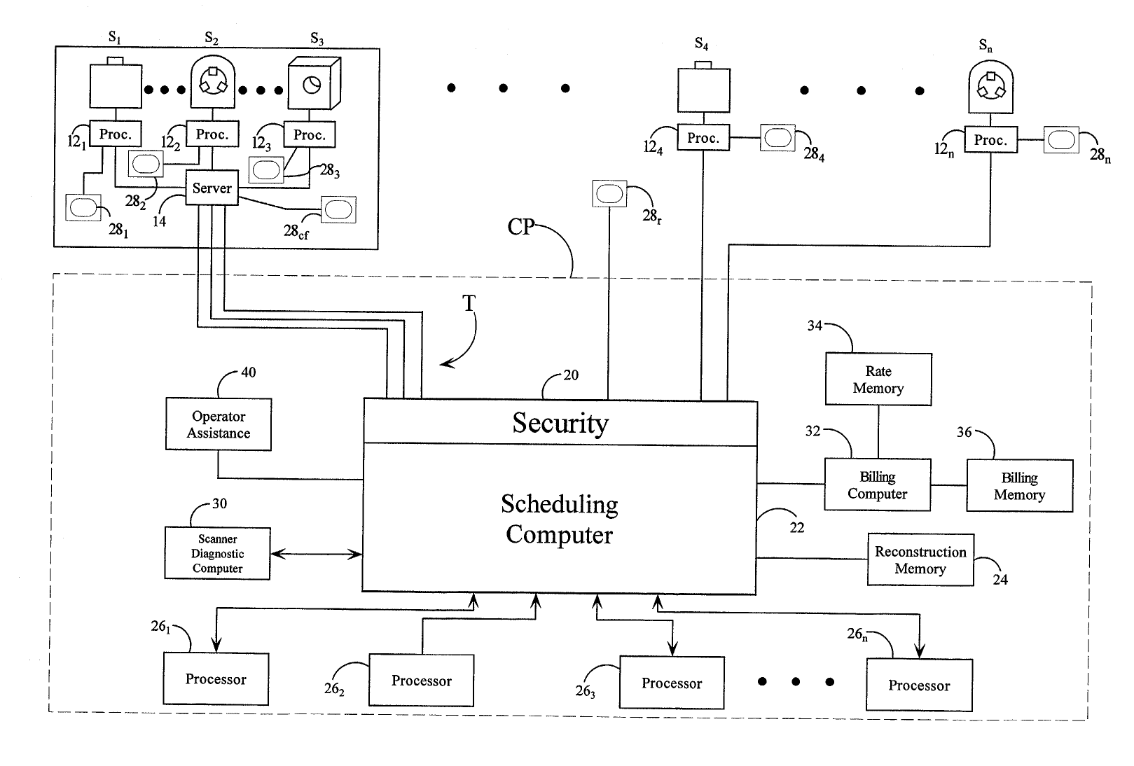

### BACKGROUND OF THE INVENTION

The present invention relates to the diagnostic imaging arts. It finds particular application in conjunction with reconstructing diagnostic imaging scans from scanners of many modalities and will be described with particular reference thereto. It will be appreciated, however, that the invention is also applicable to single modality image systems, imaging systems in combination with non-imaging diagnostic systems, and the like.

Today, in the field of medical imaging, diagnostic imagers are widely used to generate images of the interior of a subject. Typically, medical diagnostic imagers non-invasively examine the interior of a subject with radiation, electromagnetic fields, ultrasound, and the like. Each medical imager has its own computer that controls the examination and reconstructs the resultant data to generate human-readable images of interior regions of the subject. The process of reconstruction is often very time consuming, and can last for hours after the data is gathered, depending on factors such as the modality, method of reconstruction, the size of the imaging region, resolution, and selected image quality and filtering, among other factors. A good quality, high resolution nuclear camera image reconstruction often takes 1-2 hours, with a highly sophisticated reconstruction taking as long as 16 hours on a 1.5 gigahertz Pentium 4™ processor. Although the patient examination times are significantly shorter, the long reconstruction times limit the number of patients that can be examined. Conversely, the long reconstruction times sometimes leads to the selection of faster, but less appropriate image reconstruction techniques.

Periodically, software upgrades are developed to improve the reconstruction capability. Such upgrades may involve new data collection sequences, new reconstruction algorithms, upgraded reconstruction techniques, and the like. Often, such upgrades are developed by the manufacturer of the imager and made available to purchasers of the imagers. Typically in nuclear imaging, upgraded operating and reconstruction systems are loaded on a bundle of compact disks, typically 10s of CDS. Loading the upgrade CDS is labor-intensive and time-consuming. During the upgrading process, the scanner is not available for imaging. Depending on warrantees and service contracts, individual scanners are upgraded to varying degrees leaving a multiplicity of variations of operating systems in service and in potential need of maintenance.

Further, training, diagnosing machine malfunctions, operation of the scanner, and the like are performed on a site by site basis. Some facilities only have imaging services available during prescribed hours. After hours, no operator is on duty to handle emergency imaging needs.

The present invention provides a new and improved method and apparatus which overcomes the above-referenced problems and others.

### SUMMARY OF THE INVENTION

In accordance with one aspect of the present invention, a diagnostic imaging system is provided. A plurality of scanning apparatuses generate data sets representative of a examination of a subject disposed in its imaging region. At least one communal processing center remote from the scanning apparatuses processes the data sets into representations. An information transfer pathway transports the data sets from the scanning apparatuses to the communal processing unit and transports the image representations from the communal processing unit to at least one display console. A storage unit at the communal processing site holds data until it can be processed or transmitted.

In accordance with another aspect of the present invention, an image reconstruction system includes a gantry for examining a subject and generating a data set corresponding thereto. A computer controls the gantry to implement a selected protocol for examining the subject and reconstructs the generated data sets into an image representation using at least first selected reconstruction algorithms. A communication means transmits the data sets to a remote processing location for reconstruction into an image representation using at least second reconstruction algorithms and for receiving the reconstructed image representations therefrom.

In accordance with another aspect of the present invention, a method of diagnostic imaging is provided. A data set representative of the interior of a subject which is examined is generated. At least a portion of the data set is electronically communicated to a remote reconstruction center. At the remote center, a type of data set and the reconstruction to be performed is identified. Also at the remote center, the data set is reconstructed. The reconstructed data set is electronically communicated to a remote display console. At the display console, the reconstructed data set is converted into a human-readable format.

One advantage of the present invention resides in faster image reconstruction.

Another advantage resides in both upstream (client to central location) and downstream (central location to client) services.

Another advantage of the present invention resides in lower cost.

Another advantage resides in reduced capital costs by purchasers.

Another advantage resides in the ability to place scanning gantries in settings where it would be impractical to place entire scanners.

Another advantage resides in extended availability of imaging services, particularly in emergencies or after hours.

Another advantage resides in the capability for remote training and operation.

Another advantage resides in more rapid upgrades to all users.

Another advantage resides in simplified repair potential.

Another advantage resides in access to the central database for interpretation of images.

Another advantage resides in centralized quality control and machine diagnosis.

Yet another advantage resides in multiple levels of the quality of the reconstruction.

Still further benefits and advantages of the present invention will become apparent to those skilled in the art upon a reading and understanding of the preferred embodiments.

### BRIEF DESCRIPTION OF THE DRAWINGS

The invention may take form in various components and arrangements of components, and in various steps and arrangements of steps. The drawing is only for purposes of illustrating preferred embodiments and is not to be construed as limiting the invention.

The FIGURE is a diagrammatic illustration of a plurality of diagnostic imagers and a central reconstruction center, in accordance with the present invention.

### DETAILED DESCRIPTION OF THE PREFERRED EMBODIMENT

With reference to the FIGURE, a plurality of medical diagnostic imagers S₁, S₂, . . . , Sₙ are connected by tie lines T to a central processing center CP. The tie lines T are of a large bandwidth, multiple gigabits in the preferred embodiment.

Each of the medical diagnostic imagers includes scanning hardware 10₁, 10₂, . . . , 10ₙ and a computer or processor 12₁, 12₂, . . . , 12ₙ for controlling the scanning hardware and reconstructing the resultant data. The scanning hardware includes magnetic resonance imaging systems, nuclear cameras, CT scanners, ultrasound systems, fluoroscopy, and the like. In the preferred embodiment, the on-board processors 12₁, 12₂, . . . . , 12ₙ perform simple reconstructions such as pilot scans, low resolution images, and the like. In large facilities in which there are a plurality of scanners, each of the scanners is connected with a server 14. The server 14 interfaces between the plurality of scanners and a smaller number of tie lines. optionally, the server 14 can include sufficient computing power to perform intermediate complexity reconstructions. Further, the processing center CP might be located on-site, such as multiple scanners in a single hospital sharing a pool of processing resources.

An operator uses the processors 12₁, 12₂, . . . , 12ₙ to conduct a scan using the corresponding hardware 10₁, 10₂, . . . , 10ₙ. The operator further selects the nature of the reconstruction, the priority, and the like. Based on the requested reconstruction, the processor or the server 14 performs a reconstruction or forwards the data, a description of the scanner including the nature of the modality, the selected image processing, the priority, and the like.

At the central processing center, a security system 20 screens incoming requests and properly encodes outgoing processed images. A central or scheduling computer 22 receives all of the requests and data and arbitrates among the requests. The central computer estimates the time to complete reconstruction, assigns relative priorities, and optimizes scheduling of the central processing center's resources. When the central processing center is performing real time reconstructions, the operator sends a request for the reconstruction and the scheduling computer 22 responds with a time window within which a substantially real time reconstruction can be performed. Because the patient is at the scanner and attending physicians or diagnosticians are often present during a real time scan, real time scans are typically high priority.

Once the scheduling computer receives a request, it accesses a data reconstruction memory 24 to retrieve the appropriate reconstruction algorithms. The scheduling processor then allocates portions of the reconstruction operation and the data to one or more subsidiary processors 26₁, 26₂, . . . , 26ₙ. The processors 26 are preferably state-of-the-art, but may include computers of all levels of speed and sophistication. When the processing tasks are performed in parallel by a large multiplicity of computers, even relatively slow computers can provide a completed image in a relatively short period of time. It will be noted that upgrading the reconstruction software in the reconstruction memory 24 effectively upgrades the reconstruction process for all of the scanners S₁, S₂, . . . , Sₙ in a single operation.

Once the reconstruction is complete, the reconstructed image representation is communicated back to the scanner from which the data originated or to another specified address. The image is displayed on a display 28₁, 28₂, . . . , 28ₙ associated with each scanner S₁, S₂, . . . , Sₙ, respectively, on a display 28_{cf} in the same facility but separated from the scanner, such as a central diagnosis and viewing room, on a display 28ᵣ at a remote location such as a radiologist's office, or the like. Preferably, the scheduling processor returns the reconstructed image via the tie lines T. However, returning the information by other routes, such as hard copy images, public telephone lines, disk and overnight courier, the Internet, via high speed network connections, or the like is also contemplated. In one embodiment, the returned image representation is returned in packets which are time stamped and are otherwise encoded for assembly at the receiving end.

The raw data and the reconstructed images are forwarded to a diagnostic computer 30 which examines the raw data and the images to diagnose misadjustment and maintenance problems in the scanner which sent the data. Various defects are readily apparent through known computer diagnostic tools. For example, a channel of CT image data which is consistently dark can be indicative of a malfunctioning radiation detector, a failing amplifier, or the like. Images with ghosting or phantoms, with irregular resolution, with unexpectedly poor resolution, and the like are also indicative of hardware problems. The diagnostic computer in one embodiment schedules maintenance visits by the service technician. Optionally, the diagnosing computer attaches a message to the completed image indicating the nature of the needed maintenance. When a scanner reports intermittent malfunctions, the diagnostic computer connects, via the lines, with the reporting scanner to monitor malfunctions in real time. The diagnostic computer periodically downloads malfunction or usage listings from the individual scanner. Where appropriate, the service computer reprograms or otherwise controls individual scanners to correct problems.

A billing computer 32 receives indications of the scanner from which reconstruction techniques were received along with the nature of the reconstruction and its priority. The billing computer accesses a rate table or memory 34 which determines the charge for the requested reconstruction. The requested reconstruction and the fee are communicated to a billing memory 36 from which invoices are printed. Various billing scenarios are contemplated. For example, users may obtain the hardware at a very low lease rate and pay per scan. As another option, larger users may pre-purchase a specified number of scans per month. As another option, scanners with minimal reconstruction software might obtain routine, low priority reconstructions without charge, but incur a charge for priority and more sophisticated reconstructions.

Optionally, the central processing center further includes an operator assistance computer 40. The operator assistance program provides information to the technicians upon request concerning appropriate procedures, image diagnosis, and other such information. Optionally, the operator assistance program may also include advanced scanning protocols which are not stored on the individual scanners. Upon request, the advanced scanning protocols are transferred by the tie lines from the central processing center to the requesting scanner. In this manner, new or updated sequences are made available to users rapidly, without the need for loading on individual scanners.

Using the same connection lines T that receive requests from the individual scanners S₁ . . . Sₙ services are provided to users of the individual scanners. In the preferred embodiment, the central processing center CP has administrator access to the remote scanners S₁ . . . Sₙ. A systems administrator logs onto one of the remote systems through the operator assistance terminal 40 and assists in real time. This feature is used to diagnose technical problems with any one of the given remote scanners. This feature is enhanced by video and audio devices at the scan site, allowing the administrator and local operator to discuss symptoms (e.g. errors and the like) and possible remedies. If software is determined to be the problem, the administrator can upload replacement software over the communication pathway T.

In an embodiment where video and audio is available to the central site CP, the individual scanners S₁ . . . Sₙ are controlled remotely by a diagnostician at the central site. This feature is used during historically low use times, for example, in the middle of the night, when an operator and a diagnostician is not on duty at the scanner site. A nurse or other technician on site assists in patient positioning, comfort, and the like. Video feedback enables the central site operator to verify patient position and the like. For instance, an operator at the central site can control scanners at several hospitals in the region during the graveyard shift. Likewise, if a radiologist is not on-site and a diagnosis is required, a radiologist at the central site makes a diagnosis.

Similarly, a computer diagnosis can be made by referencing a database of old scans and comparing them to current scans looking for similar abnormalities and the like. This feature is used as a supplementary aid to assist in the diagnoses of physicians, however, fully automated diagnoses are contemplated. In the preferred embodiment, the database of images is used as a physician's reference to observe past diagnosed cases of a present case of a suspected malady. The physician compares the present case to past cases to observe similarities and dissimilarities therebetween.

Remote training sessions can be held for operators of the scanners S₁ . . . Sₙ individually or at a group of sites over the communication pathway T. Demonstrations are scheduled for each type of modality supported by the central processing site CP. For example, a magnetic resonance (MR) demonstrator schedules an MR demonstration of advanced or improved techniques. Clients of the central processing site have the option of “tuning in” that is, granting the demonstrator control of their scanner during at least portions of the presentation. The demonstrator controls all the participating scanners simultaneously. Alternately, the demonstrator does not have control of the scanners. The operator controls computer displays that are replicas of the scanner controls. The participants in the demonstration observe and interact with the demonstrator via audio/video links over the communication pathway T.

Another service provided to a client is interactive optimization of the scanner of the client. A technician at the central site, with the authorization of the client, remotely configures the client's scanner for optimal use. The technician reviews a scanner's usage log and client input and reconfigures software of the client's scanner via the communication pathway T and assists (via audio and video) the client in reconfiguring the hardware, if necessary.

A client having difficulty operating a scanner may request interactive support. For instance, if the scanner fails to operate and generates terminal error messages, a remote administrator logs onto the client's system via the communication pathway T, observes the problem, and assists the client in solving the problem. Such assistance is in the form of verbal instructions as well as interactive reconfiguration of aspects of the client's scanner.

It is to be understood that several of many possible services that can be provided to a client have been described. Further services will occur to those skilled in the art that fall within the scope of the claimed invention.

The invention has been described with reference to the preferred embodiment. Modifications and alterations will occur to others upon a reading and understanding of the preceding detailed description. It is intended that the invention be construed as including all such modifications and alterations insofar as they come within the scope of the appended claims or the equivalents thereof.

Having thus described the preferred embodiments, the invention is now claimed to be:

1. A diagnostic imaging system comprising:
a plurality of scanning apparatuses which each generate data sets representative of an examination of a subject disposed in an imaging region of the apparatus;
at least one communal processing center remote from the scanning apparatuses that processes the data sets into image representations of the subject;
an information transfer pathway which transports the data sets from the scanning apparatuses to the communal processing center and transports the image representations from the communal processing unit to a display console.

2. The diagnostic imaging system as set forth in claim 1, wherein the communal processing center includes:
a plurality of computers among which the data sets are divided for parallel processing.

3. The imaging system as set forth in claim 2, wherein the communal processing center includes:
a scheduling computer that assigns a processing priority to data sets and schedules.

4. The diagnostic imaging system as set forth in claim 1, wherein the communal processing center includes at least one supercomputer that processes the data sets.

5. The diagnostic imaging system as set forth in claim 1, wherein the information transfer pathway includes Internet and network connections.

6. The diagnostic imaging system as set forth in claim 1, wherein the information transfer pathway includes:
a two-way dedicated hard-line connection between the scanning apparatus and the communal processing center.

7. The diagnostic imaging system as set forth in claim 1, wherein the display console is associated with the scanning apparatus that generated the reconstructed data.

8. The diagnostic imaging system as set forth in claim 1, wherein the diagnostic scanning apparatuses each include:
a reconstruction processor for processing data sets into image representations.

9. The diagnostic imaging system as set forth in claim 1 wherein the communal processing center includes a diagnostic computer which analyzes at least one of:
the data sets,
the reconstructed image representations,
malfunction logs,
usage logs,
real-time scanning operations, and determines at least one of maintenance, repair, upgrade, reprogramming, and hardware modification procedures to be performed on each imaging apparatus.

10. The diagnostic imaging system as set forth in claim 1 wherein the communal processing center further includes:
a billing processor which receives an identification of the scanner requesting each reconstruction, a priority of the requested reconstruction, and a nature of the reconstruction, the billing computer accessing a rate table and generating a corresponding bill.

11. The diagnostic imaging system as set forth in claim 1 wherein the central processing center further includes an operator assistance computer which supplies at least one of imaging protocols, demonstrations, after hours control functions, software modifications, remote control, and demonstration services to individual scanning apparatus.

12. The diagnostic imaging system as set forth in claim 1, wherein the communication pathway provides a specialist at the remote center access to at least some functions of a selected one or more scanning apparatus for at least one of:
demonstrating functioning of the scanning apparatus;
monitoring functioning of the scanning apparatus;
operating the scanning apparatus to perform diagnostic scans on subjects in the scanning apparatus;
repairing the scanning apparatus; and,
upgrading the scanning apparatus.

13. An image reconstruction system comprising:
a gantry for examining a subject and generating a data set corresponding thereto;
a computer means for controlling the gantry to implement a selected protocol for examining the subject and for reconstructing the data sets into an image representation using at least first selected reconstruction algorithms;
a communication means for transmitting the data sets to a remote processing location for reconstruction into an image representation using at least second reconstruction algorithms and for receiving the reconstructed image representations therefrom.

14. The image reconstruction system as set forth in claim 13, further including:
a second gantry for non-invasively examining an interior of a subject;
a second computer means for controlling imaging protocols with the second gantry;
a server connected with the first and second computers for interfacing between the first and second computers and the remote processing location for controlling the sending of data sets and the receipt of reconstructed image representations.

15. A method of diagnostic imaging comprising:
generating a data set representative of an interior of a subject which is examined with a diagnostic scanning apparatus;
electronically communicating at least a portion of the data set to a remote reconstruction center;
at the remote center, identifying a type of data set and the reconstruction to be performed;
at the remote center, reconstructing the data set;
electronically communicating the reconstructed data set to a remote display console that is remote from the reconstruction center;
at the remote display console, displaying at least the reconstructed data set in a human readable format.

16. The method as set forth in claim 15, further including:
sharing resources of the remote center among a plurality of diagnostic imaging apparatuses in different facilities.

17. The method as set forth in claim 16, further including:
modifying software at the remote center to improve reconstruction for the plurality of diagnostic imaging apparatuses.

18. The method as set forth in claim 16, wherein the step of reconstructing includes:
dividing the portions of the data sets among a plurality of computers and processing them in parallel.

19. The method as set forth in claim 16, further including:
assigning a priority to data sets received at the remote center; and,
processing the data sets in accordance with the assigned priority.

20. The method as set forth in claim 16, further including:
at the remote center, analyzing at least one of the reconstructed data sets, malfunction logs of individual scanning apparatuses, usage logs, real-time scanning operations; and,
from the remote center, for individual scanning apparatuses, electronically adjusting hardware, reprogramming software, and scheduling maintenance services.

21. The method as set forth in claim 16 further including:
reconstructing some data sets at the diagnostic imaging apparatus and electronically communicating other data sets to the remote center for reconstruction into an image representation.

22. The method as set forth in claim 16 further including:
based on requested reconstruction processes, priority, and customer information, accessing a scan rate table and generating an invoice for a requested reconstruction.

23. The method as set forth in claim 16, further including:
scheduling a training session for clients of the remote center concerning a supported imaging modality;
from the remote center, accessing at least some functions of at least one scanning apparatus of the supported imaging modality and demonstrating functioning of the scanning apparatus.

24. The method as set forth in claim 16, further including:
operating individual scanning apparatus from the remote center.
